(19) United States
(12) Patent Application Publication
(10) Pub. No.: US 2019/0160003 A1
(43) Pub. Date: May 30, 2019

(54) COMPOSITIONS AND METHODS FOR TREATMENT OF INFLAMMATION OR INFECTION OF THE EYE

(71) Applicant: Veloce BioPharma, LLC., Fort Lauderdale, FL (US)

(72) Inventors: Joseph Capriotti, Puerto del Carmen (ES); Kara Capriotti, Ft. Washington, PA (US); Jesse Pelletier, Miami, FL (US)

(73) Assignee: Veloce BioPharma, LLC, Ft. Lauderdale, FL (US)

(21) Appl. No.: 16/098,979

(22) PCT Filed: May 5, 2017
(86) PCT No.: PCT/US2017/031390

§ 371 Date: Nov. 5, 2018

Related U.S. Application Data
(60) Provisional application No. 62/332,306, filed on May 5, 2016.

(51) Int. Cl. A61K 9/00; A61K 9/06; A61K 31/79; A61K 47/20; A61K 47/38; A61K 31/573; A61K 31/57

(52) U.S. Cl. CPC A61K 9/0048; A61K 9/06; A61K 31/79; A61K 31/57; A61K 47/38; A61K 31/573; A61K 47/20

(57) ABSTRACT

Compositions and methods for preventing, treating or ameliorating a condition or disorder of the eye or area surrounding the eye are disclosed and described.

### BACKGROUND OF THE INVENTION

Topical corticosteroids are routinely used to control ocular inflammation. Their mechanism of action involves the inhibition of the immune response and the subsequent tissue destruction that exuberant inflammation may cause. Corticosteroid has the undesirable side effect of limiting the body's intrinsic ability to fight infection. In fact, inopportune steroids usage can worsen the course of an infection secondary to bacteria, mycobacteria, virus, or fungus.

Thus, the use of a combined antimicrobial-steroid medication in ocular infections is recommended only under careful observation of a trained ophthalmologist because of these significant risks. In fact, TOBRADEX (Alcon), the most commonly prescribed combination ophthalmic antimicrobial-steroid drug, specifically lists ‘viral disease of the cornea and conjunctiva, mycobacteria infection, and fungal infection’ as absolute contraindications to its use.

Povidone-iodine (PVP-I) is a well-known antiseptic. There is no known antibiotic, fungal or viral resistance to PVP-I and no known species of yeast or fungus that cannot be eliminated with PVP-I. PVP-I has also been shown to inhibit the formation of biofilms and to eliminate biofilms that have already formed.

In U.S. Pat. No. 7,767,217, it is shown that under certain specific conditions, dexamethasone can be combined with povidone-iodine (PVP-I) to form an effective antimicrobial-steroid pharmaceutical solution. However, it is also shown that most preparations which combine PVP-I (or iodine) with a steroid suffer from instability due, in part, to reactivity of the iodine with the steroid. In fact, U.S. Pat. No. 3,886,268 demonstrates the well-known instability of steroid-iodine combinations. The '217 patent, and its progeny, do not disclose the use of DMSO as a solvent in any of the described compositions, and do not disclose a topical gel formulation comprising povidone-iodine and a corticosteroid with DMSO and a gelling agent.

It is widely taught that large molecules like povidone-iodine can only act on surfaces and cannot be used to penetrate skin and/or skin structures. Certain organic solvents are known to enhance the percutaneous absorption of medicaments, including dimethyl sulfoxide (DMSO). However, DMSO has, in the past, been used and described only as a transportation enhancer for small molecules and low molecular weight (LMW) drugs. It has been widely taught for decades that large, charged molecules, high-molecular weight (HMW) substances and polymers could not be effectively transported across membranes by using DMSO as a penetration enhancer. One skilled in the art would not normally employ DMSO to enhance skin penetration of large molecules, polymers or charged, high-molecular weight substances, such as povidone-iodine.

Topical formulations comprising DMSO as a penetration enhancer for small molecules are known. For example, U.S. Pat. No. 7,462,362 to Kepka, and U.S. Pat. No. 6,391,879 to Reeves, describe antifungal compositions comprising a conventional antifungal active ingredient, such as the squalene epoxidase inhibitors, terbinafine (an allylamine) or tolnaftate (a thiocarbamate), for topical administration and treatment of skin or nail fungal infection. The subject composition is free of conventional antifungal active ingredients such as the squalene epoxidase inhibitors.

Tarrand, in U.S. Pat. No. 8,512,724 describes a topical skin antiseptic composition, but is intended to not penetrate the skin and requires less than about 25% DMSO in the formulations. Although povidone-iodine is mentioned as a potential antiseptic in the formulations, there is no mention of anti-inflammatories, such as a corticosteroid. The subject composition comprises povidone-iodine, a corticosteroid, and DMSO in a topical gel formulation.

A foam formulation has also been described by Friedman in Publication No. US 2007/0292359, which requires the presence of polypropylene glycol alkyl ether. The subject composition is a stable, topical gel formulation which is not a foam and does not include polypropylene glycol alkyl ether. Yet a further composition comprising DMSO and povidone iodine is an injectable, oil-in-water dispersion described in US Pub. No. US 2003/0049320 to Bhagwatwar, et al. The current composition is not an injectable composition, and comprises povidone-iodine and corticosteroid dissolved in DMSO; it is not an oil-in-water dispersion.

More recently, the inventors of the subject invention have described topical compositions comprising povidone-iodine in DMSO. See for example, US Publication Nos. 2014/0205559 and 2015/0335676. However, these compositions are steroid-free and therefore do not include an anti-inflammatory agent useful for treating inflammation associated with ophthalmic infections. Accordingly, the stability disadvantages for the steroid in the presence of povidone-iodine or DMSO are not contemplated or addressed by these formulations.

It is surprisingly shown for the first time that DMSO can be used to enhance the efficacy and skin penetration of a large molecule like povidone-iodine, as well as a corticosteroid, when used to treat viral, fungal, or bacterial skin infections and demodex infections of the eye and eyelid, and wherein said formulation is highly stable at room temperature.

SUMMARY OF THE INVENTION

The invention relates to stable topical gel formulations useful in the treatment of inflammation or infection of the eye, including but not limited to viral infection, fungal infection, demodex infection and bacterial infection of the eyelid, skin or tissue surrounding the eye, and ocular or corneal tissue. The invention further concerns a method of treating inflammation or infection of the eye, including but not limited to viral infection, fungal infection, demodex infection and bacterial infection of the eyelid, skin or tissue surrounding the eye, and ocular or corneal tissue. In particular, the subject invention includes gel compositions comprising povidone-iodine and a corticosteroid in DMSO, wherein the compositions can be useful for treatment of viral or bacterial conjunctivitis, blepharitis, or infectious conical ulcers.

This invention discloses the surprising discovery of a novel and highly stable gel composition comprising povidone-iodine, at least one corticosteroid, and DMSO for treating infection of the eye or tissue surrounding the eye, including the eyelid.

It is surprisingly shown that the composition of the subject invention is highly stable at room temperature. The subject composition is further advantageous in that the therapeutically effective amount of steroid can be lower than typically used for ophthalmic compositions indicated for the same treatment. A gel formulation comprising, for example, 0.25% PVP-I and from 0.25% to 1.0% prednisolone which can be dissolved within the gel formulation is highly stable at room temperature for extended periods of time, comparatively longer than other corticosteroid compositions comprising higher concentrations of the steroid and higher concentrations of the povidone-iodine. Although not tied to any particular theory, the gel formulation comprising DMSO is believed to (1) increase the solubility of the salt-free steroid components in the gel formulation, or (2) prevent or reduce reactions between the steroid and povidone-iodine such that stability of both components is increased, especially the stability of the composition at room temperature.

The gel composition of the invention can advantageously be stored in HDPE or LDPE containers even at low concentration.

The DMSO in the composition of the subject invention has no known toxicity on the ocular surface when administered as part of the subject gel composition.

In addition, the gel formulation of the subject composition is non-irritating to the ocular tissue or tissue surrounding the eye.

In addition, the low-dose PVP-I (i.e., about 0.25% w/w) and steroid combinations are stable at a relatively more neutral pH (about 3.5-6) than corresponding aqueous preparations of low-dose PVP-I which require significantly more acidic (pH ranges of 2-3.4) conditions in order to preserve stability.

The present invention employs a combination of a) an antiseptic, hick is povidone iodine; b) a non-ionic form of a steroid anti-inflammatory, which in one non-limiting example is prednisolone and/or the salt form of a steroid anti-inflammatory, which in one non-limiting example is prednisolone acetate and c) dimethyl sulfoxide (DMSO). This previously unknown combination can advantageously deliver an effective therapeutic dose of a medicament to the eye for treatment of inflammation or infection of the eye, including but not limited to demodex infection and bacterial infection of the eyelid, skin or tissue surrounding the eye, and ocular or corneal tissue. In particular, the subject invention includes gel compositions comprising povidone-iodine and a corticosteroid in DMSO, wherein the compositions can be useful for treatment of viral or bacterial conjunctivitis, blepharitis, or infectious conical ulcers.

In an embodiment, disclosed herein is an ophthalmic gel composition suitable for topical administration to an eye, effective for treatment and/or prophylaxis of a microorganism infection or a disorder of at least one tissue of the eye, comprising povidone-iodine in a concentration between 0.01% and 10%, or 0.1%-5%, or 0.15%-0.75%, or 0.25%-0.60% and a steroid, e.g., a corticosteroid or its salt or ester, selected from the group consisting of prednisolone or prednisolone acetate, loteprednol etabonate, difiuprednate, and combinations thereof. In an embodiment, the povidone-iodine is between 0.1% and 2.5% by weight. In an embodiment, the povidone-iodine is between 0.5% and 2% by weight. In an embodiment, the total weight of the povidone-iodine and the steroid is between 0.1% and 4.5% in the solution. In an embodiment, the steroid is at a concentration of between 0.01 and 2%. In an embodiment, the steroid is at a concentration of between 0.05 and 1%. A preferred embodiment of the formulation can be a gel composition comprising greater than 30% DMSO; 0.25% povidone-iodine; and a corticosteroid selected from 1.0% prednisolone or a salt or ester thereof; 0.50% difluprednate, or a salt or ester thereof; and 0.5% loteprednol or a salt or ester thereof; an optional co-solvent, and a gelling went, wherein the co-solvent and gelling agent make up the balance of the composition.

In an embodiment, disclosed herein is a pharmaceutical composition comprising povidone-iodine in a concentration between 0.01% and 10%, and a steroid selected from the group consisting of prednisolone acetate, loteprednol etabonate, difluprednate, and combinations thereof, wherein the steroid is at a concentration of between 0.05 and 1%. In an embodiment, the PNIP-1. is at a concentration of about 0.25%. In an embodiment, the steroid is at a concentration selected from the group consisting of about 1.0%, about 0.50%, about 0.25%, 0.1%, about 0.05% and about 0.005%.

In an embodiment, an ophthalmic composition further comprises a composition that is free of a topical anesthetic because, advantageously, the subject composition is non-burning and non-irritating to the ocular surface and skin surrounding the eye.

In an embodiment, an ophthalmic composition is further free of an antimicrobial preservative.

In an embodiment, an ophthalmic composition optionally further comprises a co-solvent, surfactant, emulsifier or wetting agent. In an embodiment comprising a co-solvent, surfactant, emulsifier or wetting agent, the agent can be selected from the group consisting of polysorbate 20, polysorbate 60, polysorbate 80, Pluronic F-68, Pluronic F-84, Pluronic P-103, cyclodextrin, tyloxapol and the like, and combinations thereof. In an embodiment comprising co-solvent, surfactant, emulsifier or wetting agent, the agent can be provided at a concentration of about 0.01% to 2% by weight in said composition.

In an embodiment, an ophthalmic composition further comprises viscosity increasing, or gelling agent. In an embodiment, the viscosity increasing agent is selected from the group consisting of polyvinyl alcohol, polyvinylpyrrolidone, or a cellulosic gelling agent such as methyl cellulose, hydroxypropyl methylcellulose, hydroxyethyl cellulose, carboxymethylcellulose, hydroxy propyl cellulose, or the like, and a combination thereof. In an embodiment, the viscosity increasing agent is at a concentration of about 0.01% to 5% by weight in said solution.

In an embodiment, an ophthalmic composition is in the form of a solution, suspension, emulsion, ointment, cream, gel, or a controlled-release/sustain-release vehicle.

In an embodiment, a microorganism treated or prevented by prophylaxis using a composition encompassed herein is selected from the group consisting of bacteria, viruses, fungi, and amoebae. In an aspect, bacteria is mycobacteria.

In an embodiment, a disorder treated using an ophthalmic composition encompassed herein is selected from the group consisting of a microorganism infection of at least one tissue of the eye, including but not limited to viral or bacterial conjunctivitis, blepharitis, conical abrasion, corneal ulcer, ulcerative infectious keratitis, epithelial keratitis, stromal keratitis and herpesvirus-related keratitis.

In an embodiment, an ophthalmic composition is used for prophylaxis of infection following corneal abrasion or ocular surgery.

In an embodiment, an ophthalmic composition comprises:
0.1 to 1% (w/w) polyvinylpyrrolidone-iodine;
0.05 to 2% (w/w) steroid;
greater than 30% DMSO; and
0.1 to 5.0% (w/w) hydroxyethylcellulose;
   wherein the steroid is selected from the group consisting of prednisolone acetate, loteprednol etabonate, difluprednate, and combinations thereof.

In an embodiment, an ophthalmic composition comprises:
0.25% (w/w) polyvinylpyrrolidone-iodine;
0.05 to 1% steroid;
greater than 30% DMSO; and
0.1 to 5.0% (w/w) hydroxyethylcellulose;
   wherein the steroid is selected from the group consisting of prednisolone acetate, loteprednol etabonate, difluprednate, and combinations thereof.

In an embodiment, an ophthalmic composition comprises:
0.25 (4) (w/w) polyvinylpyrrolidone-iodine;
1% (w/w) prednisolone or salt or ester thereof;
greater than 30% DMSO; and
0.1 to 5.0% (w/w) hydroxyethyl cellose

In an embodiment, an ophthalmic composition comprises:
0.25% (w/w) polyvinylpyrrolidone-iodine;
0.05% (w/w) difluprednate or a salt or ester thereof;
greater than 30% DMSO; and
0.1 to 5.0% (w/w) hydroxyethylcellulose.

In an embodiment, an ophthalmic composition comprises:
0.25% (w/w) polyvinylpyrrolidone-iodine;
0.50% (w/w) loteprednol or a salt or ester thereof;
greater than 30% DMSO; and
0.1 to 5.0% (w/w) hydroxyethylcellulose.

In an embodiment, an ophthalmic composition retains 95% of its polyvinylpyrrolidone-iodine and 95% of its steroid after a period of 1 month. In an embodiment, an ophthalmic composition retains 90% of its polyvinylpyrrolidone-iodine and 90% of its steroid after a period of 3 months. In an embodiment, an ophthalmic composition retains 90% of its polyvinylpyrrolidone-iodine and 90% of its steroid after a period of 1 month.

In an embodiment, an ophthalmic composition comprising polyvinylpyrrolidone-iodine (PVP-I) and at least one steroid retains about 89% of its PVP-I after a period of 1 month, about 90% of its PVP-I after a period of 1 month, about 91% of its PVP-I after a period of 1 month, about 92% of its PVP-I after a period of 1 month, about 93% of its PVP-I after a period of 1 month, about 94% of its PVP-I after a period of I month, about 94% of its PVP-I after a period of 1 month, about 95% of its PVP-I after a period of 1 month, about 96% of its PVP-I after a period of 1 month, about 97% of its PVP-I after a period of 1 month, about 98% of its PVP-I after a period of 1 month, or about 99% of its PVP-I after a period of 1 month.

In an embodiment, an ophthalmic composition comprising polyvinylpyrrolidone-iodine (PVP-I) and at least one steroid retains about 89% of its PVP-I after a period of 3 months, about 90% of its PVP-I after a period of 3 months, about 91% of its PVP-I after a period of 3 months, about 92% of its PVP-I after a period of 3 months, about 93% of its PVP-I after a period of 3 months, about 94% of its PVP-I after a period of 3 months, about 94% of its PVP-I after a period of 3 months, about 95% of its PVP-I after a period of 3 months, about 96% of its PVP-I after a period of 3 months, about 97% of its PVP-I after a period of 3 months, about 98% of its PVP-I after a period of 3 months, or about 99% of its PVP-I after a period of 3 months.

In an embodiment, an ophthalmic composition comprising PVP-I and at least one steroid retains about 89% of its at least one steroid after a period of 1 month, about 90% of its at least one steroid after a period of 1 month, about 91% of its at least one steroid after a period of 1 month, about 92% of its at least one steroid after a period of 1 month, about 93% of its at least one steroid after a period of 1 month, about 94% of its at least one steroid after a period of 1 month, about 94% of its at least one steroid after a period of 1 month, about 95% of its at least one steroid after a period of 1 month, about 96% of its at least one steroid after a period of 1 month, about 97% of its at least one steroid after a period of 1 month, about 98% of its at least one steroid after a period of 1 month, or about 99% of its at least one steroid after a period of 1 month.

In an embodiment, an ophthalmic composition comprising PVP-I and at least one steroid retains about 89% of its at least one steroid after a period of 3 months, about 90% of its at least one steroid after a period of 3 months, about 91% of its at least one steroid after a period of 3 months, about 92% of its at least one steroid after a period of 3 months, about 93% of its at least one steroid after a period of 3 months, about 94% of its at least one steroid after a period of 3 months, about 94% of its at least one steroid after a period of 3 months, about 95% of its at least one steroid after a period of 3 months, about 96% of its at least one steroid after a period of 3 months, about 97% of its at least one steroid after a period of 3 months, about 98% of its at least one steroid after a period of 3 months, or about 99% of its at least one steroid after a period of 3 months.

In an embodiment, an ophthalmic composition is a homogeneous gel and the active ingredients, namely the povidone-iodine and steroid, are dissolved in the composition and are not an oil-in-water dispersion.

In an embodiment, a method is provided for treating and/or prophylaxis of an eye disorder or a microorganism infection of at least one tissue of the eye comprising the step of administering one of more doses of an ophthalmic composition encompassed herein to the eye. In an embodiment, the prophylaxis is prophylaxis of infection following corneal abrasion or ocular surgery. In an embodiment, the eye disorder is selected from the group consisting of a microorganism infection of at least one tissue of the eye, viral of bacterial conjunctivitis, blepharitis, corneal abrasion, corneal ulcer, ulcerative infectious keratitis, epithelial keratitis, stromal keratitis and herpesvirus-related keratitis. In an embodiment, the microorganism is a bacteria, virus, fungi, or amoebae. In an embodiment, the bacteria is mycobacteria.

In an embodiment, in a method of treatment, the sum of the povidone-iodine and the steroid is between 0.001 mg to 5 mg per dose. In an embodiment, in a method of treatment, each dose is between 10 microliters to 200 microliters. In an embodiment, in a method of treatment, each dose is between 50 microliters to 80 microliters. In an embodiment, in a method of treatment, the administering step comprises administering a composition encompassed herein to an eye one to four times a day. In an embodiment, in a method of treatment, the administering step comprises administering a composition encompassed herein to an eye one to twenty-four times a day. In an embodiment, in a method of treatment, the method includes storing the composition for at least one month, at least three months, at least six months, or at least 1 year before the administration step.

### DETAILED DESCRIPTION OF THE INVENTION

The present invention incorporates an anhydrous penetration enhancer, e.g., DMSO, and an iodophor, preferably povidone-iodine (PVP-I), and a steroid. The invention is surprisingly useful for the treatment of conditions afflicting the eye or tissue surrounding the eye, such as viral or bacterial conjunctivitis, blepharitis, and infectious corneal ulcers, demodex infection and infection of the skin surrounding the eye or eyelids.

A specific but non-limiting example of a formulation that leads to a useful pharmaceutical preparation consists of solid PVP-I and a steroid, e.g., a corticosteroid, dissolved in DMSO, and formulated using a gelling agent to provide a gel composition having a high degree of stability at room temperature.

In another embodiment, DMSO can be added to aqueous solutions of PVP-I. In an example DMSO can be present as a co-solvent with water in the range of 10%-99%. One embodiment of such a formulation could include a range of excipients such as sodium chloride, sodium dihydrogen phosphate monohydrate, disodium hydrogen phosphate anhydrous and water, as well as others known to those skilled in the art.

Percentages set forth herein are weight/weight (w/w), with respect to the specified component in the overall composition, unless otherwise indicated. For example, a composition comprising 0.25% PVP-I and 45% DMSO has 0.25% PVP-I by weight, with respect to the total weight of the composition.

In an embodiment, a composition comprises povidone-iodine in the range of about 0.01% to about 15%. In another embodiment, a composition comprises povidone-iodine in the range between 0.05% and 12.5%. In another embodiment, a composition comprises povidone-iodine in the range between 0.05% and 10.0%. In another embodiment, a composition comprises povidone-iodine in the range between 0.1% and 10.0%. In another embodiment, a composition comprises povidone-iodine in the range between 0.1% and 5.0%. In another embodiment, a composition comprises povidone-iodine in the range between 0.25% and 9.0%. In another embodiment, a composition comprises povidone-iodine in the range between 0.2% and 2.5%. In another embodiment, a composition comprises povidone-iodine in the range between 0.5% and 7.5%. %. In another embodiment, a composition comprises povidone-iodine in the range between 0.5% and 1.0%. In another embodiment, a composition comprises povidone-iodine in the range between 0.75% and 5.0%, and in yet another embodiment, between 1.0% and 4.0%. In an embodiment, a composition comprises povidone-iodine in the range of about 0.1% to about 2.5%, about 0.2% to about 2.0%, about 0.3% to about 1.0%, and about 0.4% to about 0.75%.

In an embodiment, a composition comprises povidone-iodine, or PVP-I in the range of about 0.01% to about 15%. In another embodiment, a composition comprises PVP-I in the range between 0.05% and 12.5%. in another embodiment, a composition comprises PVP-I in the range between 0.05% and 10.0%. In another embodiment, a composition comprises PVP-I in the range between 0.1% and 10.0%. In another embodiment, a composition comprises PVP-I in the range between 0.1% and 5.0%. In another embodiment, a composition comprises PVP-I in the range between 0.25% and 9.0%. In another embodiment, a composition comprises PVP-I in the range between 0.2% and 2.5%. In another embodiment, a composition comprises PVP-I in the range between 0.5% and 7.5%. %. In another embodiment, a composition comprises PVP-I in the range between 0.5% and 1.0%. In another embodiment, a composition comprises PVP-I in the range between 0.75% and 5.0%, and in yet another embodiment, between 1.0% and 4.0%. In an embodiment, a composition comprises PVP-I in the range of about 0.1% to about 2.5%, about 0.2% to about 2.0%, about 0.3% to about 1.0%, and about 0.4% to about 0.75%.

Compositions disclosed herein may comprise one or more steroids. Steroids include, but are not limited to, dexamethasone, dexamethasone alcohol, dexamethasone sodium phosphate, fluromethalone acetate, fluormethalone acetate, fluromethalone alcohol, lotoprednol etabonate, medrysone, prednisolone acetate, prednisolone sodium phosphate, difluprednate, rimexolone, hydrocortisone, hydrocortisone acetate, lodoxamide tromethamine, and any combinations thereof in an embodiment, a steroid is present in the composition at a level of about 0.001% to about 10%. In an embodiment, a steroid is present in the composition or preparation at a level of 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0%. In an embodiment, a steroid is present in the composition or preparation at a level of about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%. In an embodiment, a steroid is present in the composition or preparation at a level of about 0.001% or less, about 0.002% or less, about 0.003% or less, about 0.004% or less, about 0.005% or less, about 0.006% or less, about 0.007% or less, about 0.008% or less, about 0.009% or less, about 0.01% or less, about 0.02% or less, about 0.03% or less, about 0.04% or less, about 0.05% or less, about 0.06% or less, about 0.07% or less, about 0.08% or less, about 0.09% or less, about 0.1% or less, about 0.2% or less, about 0.3% or less, about 0.4% or less, about 0.5% or less, about 0.6% or less, about 0.7% or less, about 0.8% or less, about 0.9% or less, about 1.0% or Less, about 1.1% or less, about 1.2% or less, about 1.3% or less, about 1.4% or less, about 1.5% or less, about 1.6% or less, about 1.7% or less, about 1.8% or less, about 1.9% or less, or about 2.0% or less. In an embodiment, a steroid is present in the composition or preparation at a level of about 0.001% or more, about 0.002% or more, about 0.003% or more, about 0.004% or more, about 0.005% or more, about 0.006% or more, about 0.007% or more, about 0.008% or more, about 0.009% or more, about 0.01% or more, about 0.02% or more, about 0.03% or more, about 0.04% or more, about 0.05% or more, about 0.06% or more, about 0.07% or more, about 0.08% or more, about 0.09% or more, about 0.1% or more, about 0.2% or more, about 0.3% or more, about 0.4% or more, about 0.5%, or more, about 0.6% or more, about 0.7% or more, about 0.8% or more, about 0.9% or more, about 1.0% or more, about 1.1% or more, about 1.2% or more, about 1.3% or more, about 1.4% or more, about 1.5% or more, about 1.6% or more, about 1.7% or more, about 1.8% or more, about 1.9% or more, or about 2.0% or more.

In another embodiment, a composition comprises PVP-I at about 1%, about 2%, about 3%, about 4%, about 5%, about 6%, 7%, about 8%, about 9%, about 10%. In another embodiment, a composition comprises PVP-I at about 0.1% or less, about 0.5% or less, about 1% or less, about 2% or less, about 3% or less, about 4% or less, about 5% or less, about 6% or less, about 7% or less, about 8%, or less, about 9% or less or about 10% or less. In another embodiment, a composition comprises PVP-I at about 0.01% or more, about 0.05% or more, about 0.075% or more, about 0.1% or more, about 0.2% or more, about 0.3% or more, about 0.40 or more, about 0.5% or more, about 0.6% or more, about 0.7% or more, about 0.8% or more, about 0.9% or more, about 1% or more, about 2%, or more, about 3% or more, about 4% or more, about 5% or more, about 6% or more, about 7% or more, about 8% or more, about 9% or more or about 10% or more. In another embodiment, a composition comprises PVP-I at 0.001%, 0.005%, 0.01%, 0.05%, 0.1%, 0.2%, 0.3%, 0.4%, 0.5%, 0.6%, 0.7%, 0.0.8%, 0.9%, 1.0%, 1.25%, 1.5%, 1.75%, 7.0%, 2.25%, 2.5%, 2.75%, 3.0%, 3.5%, 4.0%, 4.5%, 5.0%, 6.0%, 7.0%, 8.0%, 9.0% or 10.0%.

In an embodiment, a composition comprises DMSO and PVP-I. In an embodiment, a composition consists essentially of DMSO and PVP-I. In an embodiment, a composition consists of DMSO and PVP-I. In an embodiment, a composition is anhydrous. In an embodiment, a composition is substantially anhydrous. In an embodiment, a composition comprises a measurable amount of water.

In an embodiment, anhydrous DIVISO is used in a composition, :In an embodiment, substantially anhydrous DMSO is used in a composition. It will be understood by one of skill in the art that DMSO can be produced and/or obtained in differing grades, and that one of the variables among DMSO preparations of different grades is the water content. By way of example, DMSO may be completely anhydrous (also referred to herein simply as “anhydrous”), substantially^{,} anhydrous, or may contain water to a measurable degree. It will be understood that the amount of measurable water in a DMSO preparation may vary based on limitations of the instrumentation and techniques used to make such measurements. In an embodiment, DMSO that is not completely anhydrous may be substantially anhydrous and contain water at a level below levels of detectability. In an embodiment, DMSO that is not completely anhydrous may contain water, wherein the water content is about at least 0.01%, about at least 0.02%, about at least 0.03%, about at least 0.04%, about at least 0.05%, about at least 0.06%, about at least 0.07%, about at least 0.08%, about at least 0.09%, about at least 0.1%, about at least 0.2%, about at least 0.3%, about at least 0.4%, about at least 0.5%, about at least 0,6%, about at least 0,7%, about at least 0.8%, about at least 0.9%, about at least 1.0%, about at least 1.5%, about at least 2.0%, about at least 2.5%, about at least 5%, about at least 7.5%, about at least 10%, about at least 12.5%, or greater. In an embodiment, DMSO that is not completely anhydrous may contain water, wherein the water content is about less than 0.01%, about less than 0.02%, about less than 0.03%, about less than 0.04%, about less than 0.05%, about less than 0.06%, about less than 0.07%, about less than 0.08%, about less than 0.09%, about less than 0.1%, about less than 0.2%, about less than 0.3%, about less than 0.4%, about less than 0.5%, about less than 0.6%, about less than 0.7%, about less than 0.8%, about less than 0.9%, about less than 1.0%, about less than 1.5%, about less than 2,0%, about less than 2.5%, about less than 5%, about less than 7.5%, about less than 10%, about less than 12.5%, or greater. It will be understood that DMSO may contain one or more other impurities in addition to water.

In an embodiment, a composition comprises povidone-iodine, a penetrant, and further comprises water. In an embodiment, a composition comprises an anyhydrous iodophor and/or an anyhydrous penetrant, and further comprises water. In an embodiment, a composition comprises PVP-I, DMSO, and further comprises water. In an embodiment, a composition comprises povidone-iodine and a penetrant, and further comprises water, wherein the water content is about at least 0.01%, about at least 0.02%, about at least 0.03%, about at least 0.04%, about at least 0.05%, about at least 0.06%, about at least 0.07%, about at least 0.08%, about at least 0.09%, about at least 0.1%, about at least 0.2%, about at least 0.3%, about at least 0.4%, about at least 0.5%, about at least 0.6%, about at least 0.7%, about at least 0.8%, about at least 0.9%, about at least 1.0%, about at least 1.5%, about at least 2.0%, about at least 2.5%, about at least 5%, about at least 7.5%, about at least 10%, about at least 12.5%, or greater. In an embodiment, a composition comprises povidone-iodine and a penetrant, and further comprises water, wherein the water content is about less than 0.01%, about less than 0.02%, about less than 0.03%, about less than 0.04%, about less than 0.05%, about less than 0.06%, about less than 0,07%, about less than 0.08%, about less than 0.09%, about less than 0.1%, about less than 0.2%, about less than 0.3%, about less than 0.4%, about less than 0.5%, about less than 0.6%, about less than 0.7%, about less than 0.8%, about less than 0.9%, about less than 1.0%, about less than 1.5%, about less than 2.0%, about less than 2.5%, about less than 5%, about less than 7.5%, about less than 10%, about less than 12.5%, or greater. In an embodiment, a composition comprises povidone-iodine and a penetrant, and further comprises water, wherein the water content is about 0.01% to about 12.5%, about 0.02% to about 10.0%, about 0.03% to about 7.5%, about 0.04% to about 5%, about 0.05% to about 2.5%, about 0.06% to about 2%, about 0.07% to about 1.5%, about 0.08% to about 1%, about 0.09% to about 0.9%, about 0.1% to about 0.8%, or about 0.2% to about 0.7%. In an aspect, the water may be derived from a component of the composition. In another aspect, the water may be specifically added to the composition.

In an embodiment, a composition comprises at least one of United States Pharmacopeial Convention (USP) grade DMSO, Active Pharmaceutical Ingredient (API) grade DMSO, analytical grade DMSO, and American Chemical Society (ACS) Spectrophotometric grade DMSO. In an embodiment, a composition comprises DMSO having <0.1% water by KF titration and >99.9% determined on an anhydrous basis.

As set forth above, the percent amount of DMSO in a composition is described in a weight-to-weight (w/w) ratio with respect to one or more other components of the composition, unless otherwise indicated. In an embodiment, the weight percent DMSO is the balance of the weight percent after addition of PVP-I. By way of a non-limiting example, a composition may comprise 1 weight percent (1%) PVP-I and 99 weight percent (99%) DMSO. It will be understood that in the foregoing example, the DMSO component of the composition may be completely anhydrous, substantially anhydrous, or may contain water to a measurable degree. In an embodiment, the weight percent DMSO is the balance of the weight percent after addition of PVP-I and any other components (e.g., co-solvent, water, additional active ingredient, etc.). In an embodiment, the weight percent DMSO is the balance of the weight percent after addition of iodophor and other components, if any. In an embodiment, the weight percent penetrant in a composition is the balance of the weight percent after addition of iodophor and other components, if any.

In an embodiment, a composition comprises DMSO in the range of 50% to 99.99%. In an embodiment, a composition comprises DMSO in the range of 1% to 99.99%. In another embodiment, a composition comprises DMSO in the range of 5% and 99.9%. In another embodiment, a composition comprises DMSO in the range of 10% and 99.9%. In another embodiment, a composition comprises DMSO in the range of 15% and 99.9%. In another embodiment, a composition comprises DMSO in the range of 20% and 99.9%. In another embodiment, a composition comprises DMSO in the range of 25% and 99.9%. In another embodiment, a composition comprises DMSO in the range of 30% and 99.9%. In another embodiment, a composition comprises DMSO in the range of 35% and 99.9%. In another embodiment, a composition comprises DMSO in the range of 40% and 99.9%. In another embodiment, a composition comprises DMSO in the range of 44% and 99.9%. In another embodiment, a composition comprises DMSO in the range of 45% and 99.9%. In another embodiment, a composition comprises DMSO in the range of 50% and 99,9%. In another embodiment, a composition comprises DMSO in the range of 55% and 99.9%. In another embodiment, a composition comprises DMSO in the range of 60% and 99.9%. In another embodiment, a composition comprises DMSO in the range of 65% and 99.9%. In another embodiment, a composition comprises DMSO in the range of 70% and 99.9%. In another embodiment, a composition comprises DMSO in the range of 75% and 99.9%. In another embodiment, a composition comprises DMSO in the range of 80% and 99.9%. In another embodiment, a composition comprises DMSO in the range of 85% and 99.9%. In another embodiment, a composition comprises DMSO in the range of 90% and 99.9%. and in yet another embodiment, between 95% and 99.9%.

In an embodiment, a composition comprises DMSO in weight percent of about at least about 25%, about 25.5%, about 26%, about 26.5%, about 27%, about 27.5%, about 28%, about 28.5%, about 29%, about 29.5%, about 30%, about 30,5%, about 31%, about 31,5%, about 32%, about 32.5%, about 33%, about 33.5%, about 34%, about 34.5%, about 35%, about 35.5%, about 36%, about 36.5%, about 37%, about 37,5%, about 38%, about 38.5%, about 39%, about 39.5%, about 40%, about 40.5%, about 41%, about 41.5%, about 42%, about 42.5%, about 43%, about 43.5%, about 44%, about 44.5%, about 45%, about 45.5%, about 46%, about 46.5%, about 47%, about 47,5%, about 48%, about 48.5%, about 49%, about 49.5%, about 50%, about 50.5%, about 51%, about 51.5%, about 52%, about 52.5%, about 53%, about 53.5%, about 54%, about 54.5%, about 55%, about 55.5%, about 56%, about 56.5%, about 57%, about 57.5%, about 58%, about 58.5%, about 59%, about 59.5%, about 60%, about 60.5%, about 61%, about 61.5%, about 62%, about 62.5%, about 63%, about 63.5%, about 64%, about 64.5%, about 65%, about 65.5%, about 66%, about 66.5%, about 67%, about 67.5%, about 68%, about 68.5%, about 69%, about 69.5%, about 70%, about 70.5%, about 71%, about 71.5%, about 72%, about 72.5%, about 73%, about 73.5%, about 74%, about 74.5%, about 75%, about 75.5%, about 76%, about 76.5%, about 77%, about 77.5%, about 78%, about 78.5%, about 79%, about 79.5%, about 80%, about 80.5%, about 81%, about 81.5%, about 82%, about 82.5%, about 83%, about 83,5%, about 84%, about 84.5%, about 85%, about 85.5%, about 86%, about 86.5%, about 87%, about 87.5%, about 88%, about 88.5%, about 89%, about 89.5%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.5%, about 99.9%, or any range determinable from the preceding amounts (for example, about 1% to about 45.5% or about 30.0% to about 49.0%).

In an embodiment, a composition comprises DMSO in weight percent of about 30%-50%; about 31%-50%; about 32%-50%; about 33%-50%; about 34%-50%; about 35%-5-%; about 36%-50%; about 37%-50%, about 38%-50%; about 39%-50%; about 40% to about 50%, about 41% to about 50%, about 42% to about 50%, about 43% to about 50%, about 44% to about 50%, about 45% to about 50%, about 46% to about 50%_{;} about 47% to about 50%, about 48% to about 50%, about 49% to about 50%, 40% to about 49%, about 41% to about 49%, about 42% to about 49%, about 43% to about 49%, about 44% to about 49%, about 45% to about 49%, about 46% to about 49%, about 47% to about 49%, about 48% to about 49%, about 40% to about 48%, about 41% to about 48%, about 42% to about 48%, about 43% to about 48%, about 44% to about 48%, about 45% to about 48%, about 46% to about 48%, about 47% to about 48%, 40% ₜo about 47%, about 41% to about 47%, about 42% to about 47%, about 43% to about 47%, about 44% to about 47%, about 45% to about 47%, about 46% to about 47%_{;} 40% ₜo about 46%, about 41% to about 46%, about 42% to about 46%, about 43% to about 46%, about 44% to about 46%. about 45% to about 46%, 40% to about 45%, about 41% to about 45%, about 42% to about 45%, about 43% to about 45%, about 44% to about 45%, 40% to about 44%, about 41% to about 44%, about 42% to about 44%, about 43% to about 44%, 40% ₜo about 43%, about 41% to about 43%, about 42% to about 43%, about 40% to about 42%, about 41% to about 42%, or about 40% to about 41%. In one embodiment, a composition comprises DMSO in weight percent of about 30% to about 50% or about 35% to about 49%, or about 40% to about 48%, or about 41% to about 45%, or about 44%. In one embodiment, a composition comprises up to 65% DMSO in weight percent. In one embodiment, a composition comprises up to 60% DMSO in weight percent. In one embodiment, a composition comprises up to 55% DMSO in weight percent. In one embodiment, a composition comprises up to 50% DMSO in weight percent. In one embodiment, a composition comprises up to 49% DMSO in weight percent. In one embodiment, a composition comprises up to 45% DMSO in weight percent. In one embodiment, a composition comprises up to 44% DMSO in weight percent.

In an embodiment, a composition comprises DMSO but does not comprise any additional solvent (e.g., co-solvent) or penetrant. In another embodiment, a composition comprises DMSO in the range of about 0.01% to 99.99% and further comprises at least one co-solvent in the range of 0.01% to about 99.99%. In an embodiment, a composition comprises DMSO and further comprises at least one co-solvent in the range of about 0.1% to about 50%. In another embodiment, a composition comprises DMSO and further comprises at least one co-solvent in the range between about 5% and about 50%. In another embodiment, a composition comprises DMSO and further comprises at least one co-solvent in the range between about 10% and about 99%. In another embodiment, a composition comprises DMSO and further comprises at least one co-solvent in the range between about 20% and about 95%. In an embodiment, a composition comprises DMSO and further comprises at least one co-solvent in the range of about 50% to about 60%, about 60?/k to about 80%, about 70% to about 90%, and about 80% to about 95%. In an aspect, water is a co-solvent. In an embodiment, a composition comprises DMSO, water, and at least one additional co-solvent. In an embodiment, a composition comprises DMSO, water, and at least two additional co-solvents. In an embodiment, a composition is substantially anhydrous and comprises DMSO and at least one additional co-solvent.

In an embodiment, a composition comprises a co-solvent in the range of 1% to 99.99%. in another embodiment, a composition comprises a co-solvent in the range of 5% and 99.9%. In another embodiment, a composition comprises a co-solvent in the range of 10% and 99.9%. In another embodiment, a composition comprises a co-solvent in the range of 20% and 99.9%. In another embodiment, a composition comprises a co-solvent in the range of 30% and 99.9%. In another embodiment, a composition comprises a co-solvent in the range of 40% and 99.9%. In another embodiment, a composition comprises a co-solvent in the range of 50% and 99.9%. In another embodiment, a composition comprises a co-solvent in the range of 60% and 99.9%. In another embodiment, a composition comprises a co-solvent in the range of 70% and 99.9%. In another embodiment, a composition comprises a co-solvent in the range of 80% and 99.9%, and in yet another embodiment, between 90% and 99.9%.

In an embodiment, a composition comprises a co-solvent at about 1%. In other embodiments, a composition comprises a co-solvent at about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 21%, about 22%, about 23%, about 24%, about 25%, about 26%, about 27%, about 28%, about 29%, about 30%, about 31%, about 32%, about 33%, about 34%, about 35%, about 36/%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

Examples of co-solvents include, but are not limited to, water, alcohols, silicones, polyethylene glycol, propylene glycol, glycerin, petrolatum, hydroxymethylcellulose, methylcellulose, and combinations thereof. In an embodiment, a co-solvent is propylene glycol. In another embodiment, a cosolvent is polypropylene glycol.

In an embodiment, a composition comprises DMSO in the range of about 0.01% to 99.99% and further comprises at least one penetrant in the range of 0.01% to about 99.99%. In an embodiment, a composition comprises DMSO and further comprises at least one penetrant in the range of about 0.1% to about 50%. In another embodiment, a composition comprises DMSO and further comprises at least one penetrant in the range between about 5% and about 50%. In another embodiment, a composition comprises DMSO and further comprises at least one penetrant in the range between about 10% and about 99%. In an embodiment, a composition comprises DMSO, at least one co-solvent, and at least one penetrant. In an embodiment, a co-solvent is also a penetrant.

In an embodiment, where possible, compositions may include pharmaceutically acceptable salts of compounds in the composition. In an embodiment, compositions comprise acid addition salts of the present compounds. In an embodiment, compositions comprise base addition salts of the present compounds. As used herein, the term pharmaceutically acceptable salts or complexes refers to salts or complexes (e.g., solvates, polymorphs) that retain the desired biological activity of the parent compound and exhibit minimal, if any, undesired toxicological effects.

In various embodiments, the compositions encompassed herein comprise pharmaceutically acceptable excipients such as those listed in REMINGTON: THE SCIENCE AND PRACTICE OF PHARMACY 866-885 (Alfonso R. Gennaro ed. 19th ed. 1995; Ghosh, T. K.; et al. TRANSDERMAL AND TOPICAL DRUG DELIVERY SYSTEMS (1997), hereby incorporated herein by reference, including, but not limited to, protectives, adsorbents, demulcents, emollients, preservatives, antioxidants_{;} moisturizers, buffering agents, solubilizing agents, skin-penetration agents, and surfactants.

Protectives and adsorbents include, but are not limited to, dusting powders, zinc sterate, collodion, dimethicone, silicones, zinc carbonate, aloe vera gel and other aloe products, vitamin E oil, allantoin, glycerin, petrolatum, and zinc oxide.

Demulcents include, but are not limited to, benzoin, hydroxypropyl cellulose, hydroxypropyl methylcellulose, and polyvinyl alcohol.

Emollients include, but are not limited to, animal and vegetable fats and oils, myristyl alcohol, alum, and aluminum acetate.

Preservatives include, but are not limited to, chlorine dioxide, quaternary ammonium compounds, such as benzalkoniutn chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin. Preferably, the subject composition is free of additional preservative.

Suitable antioxidants include, but are not limited to, ascorbic acid and its esters, sodium bisulfite, butylated hydroxytoluene, butylated hydroxyanisole, tocopherols, and chelating agents like EDTA and citric acid. Preferably, the subject composition is free of additional antioxidant.

Suitable moisturizers include, but are not limited to, glycerin, sorbitol, polyethylene glycols, urea, and propylene glycol.

Suitable solubilizing agents include, but are not limited to, quaternary ammonium chlorides, cyclodextrins, benzyl benzoate, lecithin, and polysorbates.

Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methylpyrrolidone.

In an embodiment, a composition comprises PVP-I, DMSO, a steroid, and polypropylene glycol. In an embodiment, a composition comprises 0.25% PVP-I, 44% DMSO, 1.75% HEC as a gelling agent, and 10% propylene glycol as cosolvent. In an embodiment, the composition is substantially anhydrous. In an embodiment, a composition comprises PVP-I, DMSO, hydroxymethylcellulose, propylene glycol and glycerine. In an embodiment, a composition comprises 0.1%-1.0% PVP-I, about 40%-45% DMSO, and 10-33% propylene glycol and at least one additional inactive ingredient.

In one embodiment, the composition includes 0.1-3% PVP-I, 40-49% DMSO, 8-15% alcohol, 18-25% propylene glycol, 0-2% gelling agents, and 0-3% water. In one embodiment, the composition includes aprotic solvents. In one embodiment, the composition includes 0.15-3% PVP-I, 30-50% DMSO, 10-35% propylene glycol, 0-2% gelling agents, and 0-3% water. In one embodiment, the composition includes aprotic solvents.

In one embodiment, the invention comprises DMSO 40-50%(w/w), 0.25%-5% PVP-I (w/w) and hydroxypropyl methylcellulose or hydroxymethyl cellulose or hydroxyethyl cellulose. A preferred concentration of cellulosic gelling agent is between about 1% and 3%, more preferably between about 1.5% and 2.5%, and most preferred about 1.75%. In an embodiment of the invention, a gelling agent can be provided between about 0.5% to 3.0%, between about 1.0 to 2.5%, between about 1.25 to 2.25%, between about 1.5-2.0%, or between about 1.7 to 1.8%. A preferred amount of gelling agent can be 1.75% of the total composition. A preferred gelling agent is a cellulosic gelling agent. A more preferred gelling agent is hydroxyethylcellulose (HEC).

In one embodiment, the composition is a solution; semi-solid, e.g., a gel, suspension, ointment or cream; tincture; foam; aerosol or another common pharmaceutical dosage form. In one embodiment, the composition is a 0.25% PVP-I/44% DMSO solution. In one embodiment, the composition is a 1% PVP-I/45% DMSO solution. In one embodiment, the composition is a 1.5% PVP-I/46% DIVISO solution. In one embodiment, the composition is a 2.5% PVP-I/43% DMSO solution, In one embodiment, the composition is a 1% PVP-I/99% DMSO solution. In one embodiment, the composition is a 2% PVP-I/65% DMSO solution. In one embodiment, the composition is a 2% PVP-I/65% DMSO/10-25% propylene glycol solution.

### Stability

### A. Measured as Available Iodine Remaining

In one embodiment, the formulations can be stable at room temperature 25° degrees C. for at least 1 month, 3 months, 6 months, 12 months, 18 months and 24 months. Stability is defined as where the final PVP-I concentration, measured by sodium thiosulfate titration to determine available iodine, is at least 90% of the labeled concentration (e.g. if the label is 2% PVP-I providing for 0.2% available iodine, therefore 90% would be 0.18 elemental iodine). In another embodiment, the stability as determined by sodium thiosulfate titration to determine available iodine is at least 80% at 1 month, 3 months, 6 months, 12 months, 18 months and 24 months after storage at 25 degrees C.

In one embodiment, the formulations can be stable at room temperature 2-8 degrees C. for at least 1 month, 3 months, 6 months, 12 months, 18 months and 24 months. Stability is defined as where the final PVP-I concentration is at least 90% of the labeled concentration (e.g. if the label is 2% PVP-I providing for 0.2% iodine, therefore 90% would be 0.18 elemental iodine.) In another embodiment, the stability as determined by sodium thiosulfate titration to determine available iodine is at least 80% at 1 month, 3 months, 6 months, 12 months, 18 months and 24 months after storage at 2-8 degrees C.

In one embodiment, the formulations can be stable at room temperature −10 to −25 degrees C. for at least 1 month, 3 months, 6 months, 12 months, 18 months and 24 months. Stability is defined as where the final PVP-I concentration is at least 90% of the labeled concentration (e.g. if the label is 2% PVP-I providing for 0.2% iodine, therefore 90% would be 0.18 elemental iodine). In another embodiment, the stability as determined by sodium thiosulfate titration to determine available iodine is at least 80% at 1 month, 3 months, 6 months, 12 months, 18 months and 24 months after storage at −10 to −25 degrees C.

In one embodiment, the formulations can be stable at room temperature 15-30 degrees C. for at least 1 month, 3 months. 6 months, 12 months, 18 months and 24 months. Stability is defined as where the final PVP-I concentration is at least 90% of the labeled concentration (e.g. if the label is 2% PVP-I providing for 0.2% iodine, therefore 90% would be 0.18 elemental iodine). In another embodiment, the stability as determined by sodium thiosulfate titration to determine available iodine is at least 80% at 1 month, 3 months, 6 months, 12 months, 18 months and 24 months after storage at 15-30 degrees C.

In one embodiment, the formulations can be stable at room temperature 40 degrees C. for at least 1 months, 3 months, 6 months, 12 months, 18 months and 24 months. Stability is defined as where the final PVP -I concentration is at least 90% of the labeled concentration (e.g. if the label is 2% PVP-I providing for 0.2% iodine, therefore 90% would be 0.18 elemental iodine). In another embodiment, the stability as determined by sodium thiosulfate titration to determine available iodine is at least 80% at 1 month, 3 months, 6 months, 12 months, 18 months and 24 months after storage at 40 degrees C.

### Measured as Percent Steroid Remaining

In one embodiment, the stability of the PVP-I/steroid gel combinations can be measured by determining the amount of steroid remaining after a period of time from 1 month, 2 months, 3 months, 6 months, 12 months or 24 months examining the SCF-MS for the component steroid. SCF-MS of, for example, a gel comprising 0.25% PVP-I, 44% DMSO and 1.0% prednisolone acetate shows that after at least 2 months 99.1% of the prednisolone acetate remains in the formulation and no reaction is seen between the PVP-I and the prednisolone acetate. Additionally, there is no iodination of the prednisolone acetate and no detectable formation of a PVP-I prednisolone acetate complex.

In another embodiment, the stability of the PVP-I/steroid gel combinations can be measured by determining the amount of steroid remaining after a period of time from 1 month, 2. months, 3 months, 6 months, 12 months or 24 months by examining the SCF-MS for the component steroid. SCF-MS of, for example, a gel comprising 0.25% PVP-I, 44% DMSO and 0.5% prednisolone acetate shows that after at least 2 months an average of 91% of the prednisolone acetate remains in the formulation and no reaction is seen between the PVP-I and the prednisolone acetate. Additionally, there is no iodination of the prednisolone acetate and no formation of a PVP-I prednisolone acetate complex.

In another embodiment, the stability of the PVP-I/steroid gel combinations can be measured by determining the amount of steroid remaining after a period of time from 1 month, 2 months, 3 months, 6 months, 12 months or 24 months by examining the SCF-MS for the component steroid. SCF-MS of, for example, a gel comprising 0.25% PVP-I, 44% DMSO and 0.5% prednisolone shows that after at least 2 months an average of 93% of the prednisolone remains in the formulation and no reaction is seen between the PVP-I and the prednisolone. Additionally, there is no iodination of the prednisolone and no formation of a PVP-I prednisolone complex.

### Quantitative Analysis of Hydroxyethyl Cellulose Gels Containing Prednisolone and Prednisolone Acetate

### Summary

Nineteen samples of hydroxyethyl cellulose gel containing DMSO, povidone iodine, and a steroid (either prednisolone or prednisolone acetate) were analyzed by SFC/MS after at least 2 months of storage at room temperature, atmospheric pressure and ambient humidity to confirm the presence of the steroid in the gel, to quantify the amount of steroid in the samples and to determine if any reaction occurred between the PVP-I component and the steroid component. The presence of prednisolone or prednisolone acetate was confirmed by matching the retention times and the ion exact masses to authentic samples of each steroid. The quantity of prednisolone or prednisolone acetate was determined from calibration curves based on standard solutions of known concentrations. The presence or absence of reaction between steroid and PVP-I was determined by quantifying the pure steroid component remaining after 2 months and by determining the presence or absence of iodinated steroid products in the SCF/MS.

### Materials

Test samples were prepared in the following manner:

Povidone Iodine 0.25%—Prednisolone 0.5%—DMSO 44% in Hydroxyethylcellulose Gel Formula per 100 cc of final composition
1. Weigh out 0.25 gm of Povidone Iodine, 0.5 gm of Prednisolone, 4 grams of HEC powder (per 100 cc of final composition).
2. Measure 44 mL of DMSO in a graduated cylinder.
3. In an appropriately sized container, place weighed Povidone-iodine and Prednisolone.
4. Slowly add a few drops of Polysorbate 80 into the powders and mix with a glass stirring rod until a thick paste is produced. Continue to add a few drops at a time until a loose paste is produced.
5. Slowly add DMSO while stirring until a uniform mixture is produced. Pour the rest of the DMSO into the mixture and stir with a glass stirring rod. Geometric dilution is very important in this step. Adding DMSO too fast will result in a non-homogenous mixture.
6. Qs up to 100 cc with purified water.
7. Using a magnetic spin bar and magnetic stirrer, start spinning until a funnel is formed.
8. Quickly sift the HEC into the mixture until desired consistency is achieved.

### Povidone Iodine 0.25%—Prednisolone 1.0%—DMSO 44% in Hydroxvethylcellulose Gel Formula per 100 cc of Composition

1. Weigh out 0.25 gm of Povidone Iodine, 1.0 gm of Prednisolone, 4 grams of HEC powder (per 100 cc of final composition).
2. Measure 44 ML, of DMSO in a graduated cylinder.
3. In an appropriately sized container, place weighed Povidone-Iodine and Prednisolone.
4. Slowly add a few drops of Polysorbate 80 into the powders and mix with a glass stirring rod until a thick paste is produced. Continue to add a few drops at a time until a loose paste is produced.
5. Slowly add DMSO while stirring until a uniform mixture is produced. Pour the rest of the DMSO into the mixture and stir with a glass stirring rod. Geometric dilution is important in this step. Adding DMSO too quickly will result in a non-homogenous mixture.
6. Qs up to 100 cc with purified water.
7. Using a magnetic spin bar and magnetic stirrer, start spinning until a funnel is formed.
8. Quickly sift the HEC into the mixture until desired consistency is achieved.

### Povidone Iodine 0.25%—Prednisolone Acetate 0.5%—DMSO 44% in Hydroxyethylcellulose Gel Formula per 100 cc of Composition

1, Weigh out 0.25 gm of Povidone Iodine, 0.5 gm of Prednisolone acetate, 4 grams of 1-IEC powder (per 100 cc of final composition).
2. Measure 44 mL of DMSO in a graduated cylinder.
3. In an appropriately sized container, place weighed Povidone-Iodine and Prednisolone acetate.
4. Slowly add DMSO while stirring until a uniform mixture is produced. Pour the rest of the DMSO into the mixture and stir with a glass stirring rod.
5. Qs up to 100 cc with purified water.
6. Using a magnetic spin bar and magnetic stirrer, start spinning until a funnel is formed.
7. Quickly sift the HEC into the mixture until desired consistency is achieved.

### Povidone Iodine 0.25%—Prednisolone Acetate 1.0%—DMSO 44% in Hydroxyethylcellulose Gel Formula per 100 cc of Composition

1. Weigh out 0.25 gm of Povidone Iodine, 1.0 gm of Prednisolone acetate, 4 grams of HEC powder (per 100 cc of final composition).
2. Measure 44 mL of DMSO in a graduated cylinder,
3. In an appropriately sized container, place weighed Povidone-Iodine and Prednisolone acetate.
4. Slowly add DMSO while stirring until a uniform mixture is produced. Pour the rest of the DMSO into the mixture and stir with a glass stirring rod.
5. Qs up to 100 cc with purified water.
6. Using a magnetic spin bar and magnetic stirrer, start spinning until a funnel is formed.
7. Quickly sift the HEC into the mixture until desired consistency is achieved.

Test samples of prednisolone and prednisolone acetate gels arrived after at least 2 months to the testing laboratory as, yellow-to-orange gels in capped 10-mL syringes. Test samples were further stored for up to 10 days in the dark at 4° C. and allowed to warm to room temperature before preparing quantitation solutions. Prednisolone (USP), prednisolone acetate (pharmaceutical standard traceable to USP), and dexamethasone (USP) (internal standard) were purchased from Sigma-Aldrich. Hydroxyethyl cellulose powder was purchased from Sigma-Aldrich. Hydroxyethyl cellulose gel (3% aqueous) was prepared by slow addition of the powder to LC/MS grade water with stirring. The mixture was stirred overnight at room temperature to yield a viscous, homogeneous, slightly orange gel. Water, methanol, isopropanol, and acetic acid (all LC/MS grade) were purchased from Fisher, DMSO (spectrophotometric grade) was also purchased from Fisher. Waters TruView LCMS certified vials were used in the quantitation assay.

### SFC/MS Method and Peak Identification

A method for analyzing the steroid-based samples was developed on a supercritical fluid chromatography (SFC) system that uses supercritical carbon dioxide (scCO₂) as the main eluent along with a polar additive. SFC methods for separating steroids have been reported. Mass spectrometry conditions for these and related compounds have also been reported. Using these reports as a guide, the following SFC/MS method was developed.

| | |
|---|---|
| SFC Instrument | Waters Acquity UPC |
| Eluent | 91:9 scCO₂:methanol at 1.5 mL/min |
| Column | Waters Acquity UPC² Torus 2-PIC, 130 Å, 1.7 μm, |
| | 3 × 100 mm |
| Method Length | 8.0 min |
| Temperature | 40° C. |
| ABPR Pressure | 2500 psi |
| Make-up Flow | 95:5 isopropanol:water with 0.2% acetic acid at 0.5 |
| | mL/min |
| Injection Volume | 0.5 μL |
| Autosampler | 20° C. |
| Temperature | |
| MS Instrument | Waters Xevo G2-XS QToF |
| Ion Source | APCI IonSabre II probe with LockSpray (ESI) |
| Corona Pin | 1.4 μA |
| Sampling Cone | 40 V |
| Source Temperature | 120° C. |
| Probe Temperature | 300° C. |
| Desolvation Gas | 700 L/h (nitrogen) |
| Cone Gas | 50 L/h |
| Collision Energy | 6.0 V (argon) |

As seen in FIGS. 1 and 2, the retention times of prednisolone and prednisolone acetate in the mass chromatograms are 6.6 min and 2.6 min, respectively, matching the retention times determined from authentic samples of prednisolone and prednisolone acetate. The identity of these peaks was also confirmed by mass spectrometry (MS). By MS, the peak at 6.6 min showed 361.2 m/z corresponding to [M-H]⁺ for prednisolone, and the peak at 2.6 min showed 403.2 m/z corresponding to [M+H]⁺ for prednisolone acetate.

### Standard and Sample Preparation

The method for extracting prednisolone and prednisolone acetate from the hydroxyethyl cellulose gels was modeled after a reported method for extracting methanol-soluble drug compounds from hydroxyethyl cellulose gels.

### Calibration Standards.

To mimic the steroid extraction method used in sample preparation, the calibration standards were made from a mixture of the steroid in DMSO and hydroxyethyl cellulose gel. This gel mixture was then extracted using the same method as the test samples.

Prednisolone: to a 5-mL volumetric flask was added prednisolone (10.0 mg), DMSO (42.0 mg), and hydroxyethyl cellulose (3% aqueous) gel (HEC, 44.4 mg), which was vortexed to yield a heterogeneous film containing 10.4% prednisolone (w/w). The volumetric flask was filled to 5.00 mL with methanol, and the flask was capped and placed in a sonicating bath for 1 hour to yield a solution that was 2.00 mg/mL prednisolone in methanol.

A portion of this solution was passed through a syringe filter (PTFE, 0.45-μm pore size), from which 1.00 mL was taken and diluted with 3.00 mL methanol to make a stock solution with 0.500 mg/mL prednisolone in methanol. This stock solution was diluted into a 2-mL LC/MS vial according to Table 1 to provide the prednisolone calibration standards,

TABLE 1
Preparation of calibration standards for prednisolone.

| Target | Stock | | Internal |
|---|---|---|---|
| Concentration | Solution | Methanol | Standard |
| (μg/mL) | (μL) | (μL) | (μL)^{a} |
| 400 | 800 | 170 | 30.0 |
| 300 | 600 | 370 | 30.0 |
| 200 | 400 | 570 | 30.0 |
| 100 | 200 | 770 | 30.0 |
| 80.0 | 160 | 810 | 30.0 |
| 60.0 | 120 | 850 | 30.0 |
| 50.0 | 100 | 870 | 30.0 |
| 40.0 | 80.0 | 890 | 30.0 |
| 30.0 | 60.0 | 910 | 30.0 |
| 20.0 | 40.0 | 930 | 30.0 |
| 10.0 | 20.0 | 950 | 30.0 |

^{a}dexamethasone in DMSO (0.5% w/w)

Prednisolone acetate: to a 5-mL volumetric flask was added prednisolone acetate (11.4 mg), DMSO (45.7 mg), and HEC (66.9 mg), which was vortexed to yield a heterogeneous film containing 10.1% prednisolone acetate (w/w). The volumetric flask was filled to 5.00 mL with methanol, and the flask was capped and placed in a sonicating bath for 1 hour to yield a solution that was 2.28 mg/mL prednisolone in methanol. A portion of this solution was passed through a syringe filter (PTFE, 0.45-μm pore size), from which 1.00 mL was taken and diluted with 3.56 mL methanol to make a stock solution with 0.500 mg/mL prednisolone acetate in methanol. This stock solution was diluted into a 2-mL LC/MS vial according to Table 2 to provide the prednisolone acetate calibration standards.

TABLE 2
Preparation of calibration standards for prednisolone acetate.

| | Stock | | Internal |
|---|---|---|---|
| Target Conc. | Sol'n | Methanol | Standard |
| (μg/mL) | (μL) | (μL) | (μL)^{a} |
| 400 | 800 | 150 | 50.0 |
| 300 | 600 | 350 | 50.0 |
| 200 | 400 | 550 | 50.0 |
| 100 | 200 | 750 | 50.0 |
| 80.0 | 160 | 790 | 50.0 |
| 60.0 | 120 | 830 | 50.0 |
| 50.0 | 100 | 850 | 50.0 |
| 40.0 | 80.0 | 870 | 50.0 |
| 30.0 | 60.0 | 890 | 50.0 |
| 20.0 | 40.0 | 910 | 50.0 |
| 10.0 | 20.0 | 930 | 50.0 |

^{a}dexamethasone in DMSO (0.5% w/w)

Test Sample Preparation. Approximately 1.0 g of each sample gel was added to a 50-mL volumetric flask. The flask was then filled to 50.00 mL with methanol, capped, and placed in a sonicating bath for 1 hour. An aliquot from each of these stock solutions was passed through a syringe filter (PTFE, 0.45-μm pore size), from which 500.0 μL was used for the solutions described in Tables 3 and 4 for prednisolone and prednisolone acetate, respectively.

TABLE 3
Preparation of prednisolone test samples.

| | Sample | Stock | | Internal | Expected Final |
|---|---|---|---|---|---|
| | Wt. | Sol'n | Methanol | Std | Conc. |
| Sample ID | (g) | (μL) | (μL) | (μL)^{a} | (μg/mL)^{b} |
| Pred_01 | 1.2475 | 500.0 | 470.0 | 30.00 | 62.38 |
| Pred_02 | 1.0729 | 500.0 | 470.0 | 30.00 | 53.64 |
| Pred_03 | 1.1036 | 500.0 | 470.0 | 30.00 | 55.18 |
| Pred_04 | 1.0541 | 500.0 | 470.0 | 30.00 | 52.70 |
| Pred_05 | 1.0088 | 500.0 | 470.0 | 30.00 | 50.44 |
| Pred_06 | 1.0702 | 500.0 | 470.0 | 30.00 | 53.51 |
| Pred_07 | 1.0579 | 500.0 | 470.0 | 30.00 | 52.90 |
| Pred_08 | 1.0462 | 500.0 | 470.0 | 30.00 | 104.62 |
| Pred_09 | 1.0811 | 500.0 | 470.0 | 30.00 | 108.11 |

^{a}dexamethasone in DMSO (0.5% w/w)
^{b}based on 0.5% prednisolone (w/w) sample concentration for Pred_01-07 and 1.0% prednisolone (w/w) sample concentration for Pred_08-09.

TABLE 4
Preparation of prednisolone acetate test samples.

| | Sample | Stock | | Internal | Expected Final |
|---|---|---|---|---|---|
| Sample | Wt. | Sol'n | Methanol | Std | Conc. |
| ID | (g) | (μL) | (μL) | (μL)^{a} | (μg/mL)^{b} |
| Pred- | 1.1515 | 500.0 | 450.0 | 50.00 | 57.575 |
| Ac_01 | | | | | |
| Pred- | 1.0525 | 500.0 | 450.0 | 50.00 | 52.625 |
| Ac_02 | | | | | |
| Pred- | 0.9965 | 500.0 | 450.0 | 50.00 | 49.825 |
| Ac_03 | | | | | |
| Pred- | 1.0324 | 500.0 | 450.0 | 50.00 | 51.62 |
| Ac_04 | | | | | |
| Pred- | 1.0289 | 500.0 | 450.0 | 50.00 | 51.445 |
| Ac_05 | | | | | |
| Pred- | 0.9993 | 500.0 | 450.0 | 50.00 | 99.93 |
| Ac_06 | | | | | |
| Pred- | 1.1713 | 500.0 | 450.0 | 50.00 | 117.13 |
| Ac_07 | | | | | |
| Pred- | 0.9778 | 500.0 | 450.0 | 50.00 | 97.78 |
| Ac_ 08 | | | | | |
| Pred- | 1.0379 | 500.0 | 450.0 | 50.00 | 103.79 |
| Ac_09 | | | | | |
| Pred- | 1.1547 | 500.0 | 450.0 | 50.00 | 115.47 |
| Ac_10 | | | | | |

^{a}dexamethasone in DMSO (0.5% w/w)
^{b}based on 0.5% prednisolone acetate (w/w) sample concentration for Pred-Ac_01-05 and 1.0% prednisolone acetate (w/w) sample concentration for Pred-Ac_06-10.

### Quantitation

The presence of prednisolone (Pred) or prednisolone acetate (Pred-Ac) in the test samples was verified by both SFC/MS retention time and mass spectrometry. To quantify the amount of Pred or Pred-Ac in the test samples, a calibration curve was generated from solutions of Pred or Pred-Ac with known concentrations. By comparing the integrated SFC/MS peaks of the sample solutions to the integrated SFC/MS peaks of the calibration solutions, the concentrations of the samples can be measured. To mimic the test samples when preparing the calibration standards, a stock hydroxyethyl cellulose (HEC) gel with a precisely measured amount (approx. 10% w/w final concentration in the gel) of Pred or Pred-Ac was made (see Standard and Sample Preparation section for details). This gel was then diluted to a specific volume in methanol and sonicated to break apart the gel and dissolve the steroid. This stock solution was filtered to remove any insoluble HEC, and this was diluted to varying degrees and spiked with a known amount of internal standard (dexamethasone) to provide the calibration solutions. Each calibration solution was measured on the SFC/MS instrument in triplicate.

The sample solutions were made by the same method: a measured amount of gel (by weight) was diluted to a specific volume, sonicated to break apart the gel and dissolve the steroid, filtered, spiked with a known amount of internal standard, and diluted once more to reach a concentration within the calibration range. Each sample solution was measured on the SFC/MS instrument in triplicate.

The mass chromatograms were processed using Waters' QuanLynx software to generate calibration curves and apply the calibration to the test samples. Once a sample concentration was determined, the amount of Pred or Pred-Ac in the gel could be calculated. In the prepared solutions, a gel sample weighing 1,000 g at 1.0% w/w Pred or Pred-Ac would have a final concentration of 100 μg/mL; likewise, a gel sample weighing 1.000 g that started at 0.5% w/w would have a final concentration of 50 μg/mL. if the gel sample weighed slightly more or less than 1.000 g, then the measured concentration of the prepared solution would be slightly more or less, respectively. The measured concentration could be multiplied by the correction factor (1.000 g÷gel sample weight) to provide a weight-corrected concentration (WC concentration) that can be compared and averaged with WC concentrations of other samples. The average WC concentrations for each lot of samples are given in Table 5 below, and the raw data are attached to the end of this report.

TABLE 5
Average Measured WC Concentrations for Each Lot of Samples^{a}

| | | Average/Expected | | |
|---|---|---|---|---|
| | | WC Conc. | Sid Deviation | % |
| | | (μg/mL) | μg/mL) | Difference |
| Pred_01-05: | Lot# | 50.9/50.0 | 10.6 | 1.9 |
| 05022016@3 | | | | |
| Pred_06-07: | Lot# | 42.0/50.0 | 6.1 | −16.0 |
| 04252016@16 | | | | |
| Pred_08-09: | Lot# | 86.6/100.0 | 14.0 | −13.4 |
| 04262016@10 | | | | |
| Pred-Ac_01-05: | Lot# | 44.1/50.0 | 6.8 | −11.7 |
| 05192016@6 | | | | |
| Pred-Ac_06-10: | Lot# | 99.1/100.0 | 6.7 | 0.9 |
| 05192016@7 | | | | |

^{a}A WC concentration of 50 μg/mL corresponds to 0.5% w/w concentration in the gel, and a WC concentration of 100 μg/mL corresponds to 1.0% w/w concentration in the gel.

### Possible Sources of Error

While measures were taken to reduce error in the quantitation assay, some steps are more likely than others to affect the measurements. One such source is the gel used for the calibration standards. Without access to the exact HEC gels used in formulating the test samples, a stand-in gel was made and used for the calibration standards. This gel did not contain povidone iodine and may have ratios of HEC/water/DMSO that differ from the sample gels. The stand-in gel may lead to different interactions between the gel and the steroid and different behavior in the extraction procedure; however, it is not possible to say if this would likely lead to higher or lower measured values.

An additional source of error is the extraction of the steroid from the sample gel. It is possible that some amount of steroid could be trapped in the HEC polymer at the end of the extraction process, which would lead to lower than expected concentrations. While the sonication method is reported for extracting methanol-soluble drugs from HEC gels, it was observed that longer sonication times (1 hour versus the reported 30 min) were needed to completely disintegrate the gels. After 1 hour, the sample no longer contained visible clumps of the yellow-orange gel, and instead contained white wisps of insoluble ELEC. To correct for this, the calibration standards were also extracted from HEC gels, but, as mentioned previously, the formulation of these stand-in gels is not exactly the same as that of the sample gels. In any instance this would lead to lower than expected concentrations. This is believed to be one reason that 100% recovery was not achieved in all samples and lots despite there being no evidence of reaction between steroid and PVP-I or Iodine.

### Conclusion

Samples Pred_01-09 were confirmed to contain prednisolone as the primary extracted component. Samples Pred-Ac_01-10 were confirmed to contain prednisolone acetate as the primary extracted component. Five prednisolone samples from Lot:#05022016@3 had an average measured concentration of 0.51% w/w prednisolone. Two prednisolone samples from Lot #0425201@16 had an average measured concentration of 0.42% w/w prednisolone. Two prednisolone samples from Lot# 04262016@10 had an average measured concentration of 0.87% w/w prednisolone. Five prednisolone acetate samples from Lot 05192016@6 had an average measured concentration of 0.44% w/w prednisolone acetate. Five prednisolone acetate samples from Lot# 05192016@7 had an average measured starting concentration of 0.99% w/w prednisolone acetate. The concentrations of the measured steroid components at the conclusion of 2 months' storage in ambient conditions indicates along with the absence of iodinated steroid products in the SCF/MS data indicate that no reaction between the steroid components and the PVP-I or free iodine has occurred.

### Methods Of Preparation And Use

It is known to one of skill in the art that PVP-I aqueous solutions are difficult to stabilize at low PVP-I concentrations over a long period of time. By way of a non-limiting example, at concentrations of PVP-I less than about 0.7% (w/w, aqueous), PVP-I aqueous solutions rapidly decay to yield complex mixtures of iodinated and iodine-free constituents. As described herein, it was surprisingly found that in the aprotic DMSO solvent system encompassed by the disclosure set forth herein, PVP-I solutions as low as 0.1% can be easily prepared and maintained as stable compositions for long periods of time. Also as described herein, hydrated DMSO solutions prepared from aqueous PVP-I demonstrate increased stability as noted for the PVP-I component.

In an embodiment, a composition comprises dry, solid or powdered PVP-I and a steroid or corticosteroid dissolved or suspended in a composition comprising or consisting of DMSO. In another embodiment, DMSO is added to an aqueous preparation comprising or consisting of PVP-I and a steroid. Based on the disclosure herein, one of skill in the art will understand how to prepare a composition to arrive at the desired amounts of iodine, iodophor, and DMSO, among other possible components of the compositions, such as a steroid as another active ingredient, encompassed herein.

In an embodiment, a composition is prepared by adding 10% PVP-I (w/v, aqueous) to pure DMSO q.s. to yield a resulting solution of 1% PVP-I (w/w) with DMSO. In another embodiment, compositions are prepared by dissolving solid PVP-I in pure DMSO q.s to obtain any of 0.1%, 0.2%, 0.3%, 0.4%, 0.5%, 1.0%, 1.25%, 1.5%, 2.0%, or 2.5% PVP-I (w/w), as well as about 0.1%, about 0.2%, about 0.3%, about 0.4%, about 0.5%, about 1.0%, about 1.25%, about 1.5%, about 2.0%, or about 2.5% PVP-I (w/w) compositions, with DMSO as the solvent. In yet another embodiment, compositions are prepared by dissolving solid PVP-I in pure DMSO q.s to obtain any composition set forth, described, and/or encompassed herein. Similar compositions comprising aqueous PVP-I (with and without excipients commonly used and/or known in the art) and DMSO can be prepared from a stock 10% PVP-1 aqueous solution and pure DMSO. It will be understood by the skilled artisan, however, that any starting composition of PVP-I, solid or liquid, may be used when the appropriate dilutions and adjustments are made to result in the desired final PVP-I concentration. Similarly, any starting composition of iodophor or elemental iodine may be used when the appropriate dilutions and adjustments are made to result in the desired final iodophor or elemental iodine concentration, respectively.

In an embodiment, it is particularly useful for the case of inflammatory conditions or infections of the eye or surrounding eye tissue that stable, anhydrous compositions that contain between 0.01%-10% PVP-I can be prepared in pure USP grade DMSO solvents.

It will be understood, based on the disclosure set forth herein, in view of the skill in the art, that specific dosage for compounds and compositions encompassed herein may be determined empirically through clinical and/or pharmacokinetic experimentation, and that such dosages may be adjusted according to pre-specified effectiveness and/or toxicity criteria. It will also be understood that a specific dosage and treatment regimen for any particular patient will depend upon a variety of factors, including the activity of the specific compounds employed, the characteristics of the patient, drug combination, the judgment of the treating physician and the nature and severity of the particular disease or condition being treated.

In an embodiment, a composition set forth, described, and/or encompassed herein is useful for treating one or more of—but not limited to—demodex or bacterial or viral eyelid skin infections (i.e. “blepharitis”). In an embodiment, the composition comprises PVP-I, a steroid, and DMSO with a gelling or viscosity-enhancing agent. In an embodiment, the composition consists essentially of PVP-I, a steroid, and DMSO with a gelling or viscosity-enhancing agent. In an embodiment, the composition consists of PVP-I, a steroid, and DMSO with a gelling or viscosity-enhancing agent.

In an embodiment, a therapeutic composition is prepared by optimizing one or more compounds for use in a dosage form different than that which is typically used for the compound. In an embodiment, a compound that is not typically administered in a topical dosage form is developed for use in a topical dosage form. The chemical and biological assays required for such development are known to one of skill in the art. The disclosure herein provides the skilled artisan with the guidance as to how to prepare such compounds and compositions comprising such compounds.

In an embodiment, a method of treating a subject having an an inflammatory condition or infection of the eye, cornea, or eyelid includes administration of a composition set forth, described, and/or encompassed herein to treat the eye condition or infection, including corneal ulceration, wherein the treatment can include at least one of preventing or slowing the progression of the inflammation or infection, preventing the spread of the infection, eradicating at least some of the infection, and eradicating the entire infection.

In an embodiment, a therapeutic composition is administered on a schedule at least once per month, at least once per week, two to three times per week, once a day, or multiple times per day, up to one administration per hour or 24 administrations per day. In an embodiment, a therapeutic composition is administered twice a day. In an embodiment, a therapeutic composition is administered three times a day, four times a day, five times a day, or more. In an embodiment, a therapeutic composition is administered less frequently than once a day. In an embodiment, a therapeutic composition is administered once every two days, once every three days, once every four days, once every five days, once every six days, or once every seven days. In an embodiment, a therapeutic composition is administered less frequently than once a week. In an embodiment, a therapeutic composition is administered once a month. In an embodiment, a therapeutic composition is administered twice a month.

In an embodiment, a therapeutic dosing regimen is continued for at least one day, at least two days, at least three days, at least four days, at least five days, at least six days, or at least seven days. In an embodiment, a therapeutic dosing regimen is continued for at least one week, at least two weeks, at least three weeks, at least four weeks, at least six weeks, at least eight weeks, at least ten weeks, at least twelve weeks, at least fourteen weeks, or at least sixteen weeks. In an embodiment, a therapeutic dosing regimen is continued for at least one month, at least two months, at least three months, at least four months, at least five months, at least six months, at least nine months, or at least twelve months.

The invention is further described by the following examples. In an aspect, the following examples demonstrate effective and/or successful treatment of the identified conditions using compositions and methods encompassed by the present disclosure. It should be recognized that variations based on the inventive features are within the skill of the ordinary artisan, and that the scope of the invention should not be limited by the examples. To properly determine the scope of the invention, an interested party should consider the claims herein, and any equivalent thereof. All citations herein are incorporated by reference, and unless otherwise expressly stated, all percentages are by weight/weight.

It is well described in the art that DMSO is not an effective agent to enhance the skin or tissue penetration of large (>300 MW) molecules. It is especially surprising in this invention that povidone-iodine K30 (MW>30,000 MW) was able to be transported across the heavily keratinized skin barrier through the action of DMSO. There is no clinical trial except from this current result that has shown the successful penetration of the skin barrier by povidone-iodine as the teaching has always indicated that povidone-iodine acts only on surfaces. This is an example of povidone-iodine and DMSO used together to effect skin penetration and is surprisingly successful despite decades of teaching in the art that suggests the use of DMSO should have no effect on a large molecule like povidone-iodine.

Additional examples of useful compositions described in this invention include the formulation of creams, petrolatum balms, salves, sprays, and other formulations well known to those in the art suitable for topical administration.

In additional embodiments, the PVP-I/DMSO system can be combined with a variety of naturally occurring substances and derivatives including, surprisingly, powerful anti-oxidants. Additionally, it has been found that a variety of naturopathic ingredients can be codissolved in these systems without reaction between the complexed or non-complexed iodine. A non-limiting list of such possible naturopathic additives includes Punica Granatum (Pomegranate) Extract, Camellia Sinensis Leaf (Green Tea) Extract, Ascorbic Acid (Vitamin-C), Calendula Officinalis Extract, Glycrrhiza Glabra (Licorice) Extract, Allantoin, Cucurnis Sativus (Cucumber) Fruit Extract.

It has been additionally found that the compositions described within and above can also be combined with keratolytic agents, for example urea, at concentrations below 1%, and existing anti-viral wart compounds including, for example, salicylic acid at between 0.05% and 50%, enabling further synergistic antimicrobial effect.

While the foregoing written description enables one who his ordinarily skilled in the art to reproduce and use what is considered presently to be the best mode thereof, those of ordinary skill will understand and appreciate the existence of variations, combinations, derivatives, analogs and equivalents of the specific embodiments, methods and examples provided above. The invention should therefore not be limited by the above described embodiments, examples and methods by instead by all embodiments, examples and methods within the scope and spirit of the present invention.

1. A topical ophthalmic gel composition comprising 0.1%-10% w/w povidone-iodine; greater than about 30% w/w DMSO; about 0.05% to about 1.5% w/w steroid; and a gelling agent.

2. The composition of claim 1, wherein the composition comprises a gelling agent selected from the group consisting of hydroxypropyl methylcellulose; hydroxyethyl cellulose; and hydroxymethyl cellulose.

3. The composition of claim 1, wherein the composition comprises a gelling agent selected from the group consisting of hydroxypropyl methylcellulose; hydroxyethyl cellulose; and hydroxymethyl cellulose and the gelling agent is present in a concentration between 0.5% to 3.0%, 1.0 to 2.5%, 1.25 to 2.25%, 1.5-2.0%, or 1.7 to 1.8%.

4. The composition of claim 1, wherein the composition comprises a steroid selected from the group consisting of prednisolone, difluprednate, and loteprednol or a salt or ester thereof.

5. The composition of claim 4 wherein the prednisolone is present at 1% w/w of the composition.

6. The composition of claim 4 wherein the prednisolone is present at 0.5% w/w of the composition.

7. The composition of claim 4 wherein the prednisolone acetate is present at 1% w/w of the composition.

8. The composition of claim 4 wherein the prednisolone acetate is present at 0.5% W/W of the composition.

9. The composition of claim 4 wherein the difluprednate is present at 0.05% w/w of the composition.

10. The composition of claim 4 wherein the loteprednol is present at 0.5% w/w of the composition.

11. The composition of claim 10 wherein the loteprednol is loteprednol etabonate.

12. The composition of claim 1 wherein the povidone-iodine is 0.25%-0.5% w/w of the composition.

13. A method for treating an infectious or inflammatory eye condition, said method comprising administering to the eye an effective amount of a composition of clam 1.

14. The method of claim 13 wherein the eye condition is selected from bacterial or viral conjunctivitis; blepharitis; and corneal ulcer.
